(19) United States
(12) Patent Application Publication
(10) Pub. No.: US 2023/0295080 A1
(43) Pub. Date: Sep. 21, 2023

(54) IMPROVED PROCESS FOR SYNTHESIZING FUNCTIONALIZED MERCAPTANS

(71) Applicant: ARKEMA FRANCE, Colombes (FR)

(72) Inventors: Georges FREMY, Lacq (FR); Jean-Christophe LEC, Lacq (FR)

(73) Assignee: ARKEMA FRANCE, Colombes (FR)

(21) Appl. No.: 18/005,306

(22) PCT Filed: Jul. 13, 2021
(86) PCT No.: PCT/FR2021/051300

§ 371 Date: Jan. 12, 2023

(30) Foreign Application Priority Data
Jul. 20, 2020 (FR) FR2007577
Dec. 4, 2020 (FR) FR2012669

(51) Int. Cl. C07C 319/08; C12P 13/12; C12N 9/10

(52) U.S. Cl. CPC C07C 319/08; C12N 9/1085; C12P 13/12; C12Y 205/01049

(57) ABSTRACT

The present invention relates to a process for synthesizing functionalized mercaptans essentially in the absence of oxygen, and also to a composition making it possible in particular to implement this process. Said functionalized mercaptans are of the following formula (I):

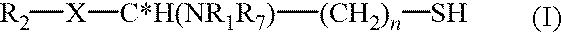

in which,
R₁ and R₇, which are identical or different, are a hydrogen atom or an aromatic or nonaromatic, linear, branched or cyclic, saturated or unsaturated, hydrocarbon chain of 1 to 20 carbon atoms which may comprise one or more heteroatoms;
X is chosen from -C(=O)-, -CH₂- or -CN;
R₂ is:
   (i) either absent when X represents -CN,
   (ii) or a hydrogen atom,
   (iii) or -OR₃, R₃ being a hydrogen atom or an aromatic or nonaromatic, linear, branched or cyclic, saturated or unsaturated, hydrocarbon chain of 1 to 20 carbon atoms which may comprise one or more heteroatoms,
   (iv) or -NR₄R₅, R₄ and R₅, which are identical or different, being a hydrogen atom or an aromatic or nonaromatic, linear, branched or cyclic, saturated or unsaturated, hydrocarbon chain of 1 to 20 carbon atoms which may comprise one or more heteroatoms;
n is equal to 1 or 2; and * represents an asymmetric carbon.

The present invention relates to a process for synthesizing functionalized mercaptans, and also to a composition making it possible in particular to implement this process.

Mercaptans are used in numerous industrial fields and many synthesis methods are known, such as the sulfhydration of alcohols, the catalytic or photochemical addition of hydrogen sulfide onto unsaturated organic compounds, or the substitution, using hydrogen sulfide, of halides, epoxides or organic carbonates.

However, these processes have many drawbacks and are not always suited to the synthesis of functionalized mercaptans, that is to say mercaptans comprising at least one functional group other than the thiol group (-SH). This type of mercaptan constitutes a chemical family with a great deal of potential, especially amino acids and derivatives with a thiol function, in particular homocysteine. They may for example be useful as synthesis intermediates for the cosmetics industry. However, there is currently no efficient synthesis method suited to the production of these functionalized mercaptans which is industrially viable, especially for applications falling under the field of commodity chemicals.

For instance, among the conventional chemical methods, substitution with hydrogen sulfide requires frequently high temperatures and pressures and leads to undesired by-products of olefin, ether, sulfide and/or polysulfide type. The catalytic or photochemical addition of hydrogen sulfide onto unsaturated compounds is generally performed under slightly milder conditions but likewise leads to many by-products formed by isomerization of the starting material, by non-regioselective addition or by double addition leading to the production of sulfides and/or polysulfides.

The main disadvantage with these conventional synthesis methods is therefore that they result in the coproduction of the mercaptan of interest and of a significant amount of associated sulfides and/or polysulfides which are difficult to upgrade. These secondary reactions lead to an increase in the variable costs associated with the starting materials because of the reduction in selectivity and hence in yield, to an increase in purification costs and to an increase in the production costs due to the expensive destruction of these by-products.

It is a known alternative to the chemical routes to synthesize functionalized mercaptans via the biological route. For example, cysteine is currently produced biologically by a fermentation route (Maier T., 2003. Nature Biotechnology, 21: 422-427). These biological routes are gentler and better suited to multifunctional molecules. But here again, the production of the mercaptan of interest is accompanied by the corresponding sulfides and/or polysulfides such as disulfides (WO 2012/053777).

There is therefore a need for an improved process for synthesizing, in particular by the biological route, functionalized mercaptans, which makes it possible in particular to limit, or even prevent, the formation of by-products such as sulfides and/or polysulfides. There is also a need for a process for synthesizing functionalized mercaptans which is safe and easy to implement industrially.

The present invention makes it possible to overcome the drawbacks of the prior art processes in whole or in part.

One objective of the present invention is to provide an improved process for synthesizing a functionalized mercaptan, in particular having a yield and/or a selectivity equivalent or superior to the known processes.

One objective of the present invention is to provide a process for synthesizing a functionalized mercaptan with negligible, or even zero, coproduction of by-products, in particular of sulfides and/or polysulfides.

The present inventors have discovered that the functionalized mercaptans of formula (I) as defined below, in particular L-homocysteine, could be advantageously synthesized by reaction between compounds of formula (II) and a hydrosulfide salt and/or a sulfide salt (hereafter denoted “salt”) as defined below or H₂S, in the presence of a sulfhydrylase enzyme, said reaction taking place essentially in the absence of oxygen, or even in the absence of oxygen.

The present inventors have thus discovered a process for synthesizing functionalized mercaptans of formula (I) which makes it possible to limit, or even prevent, the coproduction of sulfides and/or polysulfides, in particular disulfides.

More particularly, the process according to the invention makes it possible to produce L-homocysteine while at the same time limiting or even preventing the coproduction of L-homocystine and/or L-homocysteine sulfide (also called 4,4′-sulfanediylbis(2-aminobutanoic acid) / L-homolanthionine).

L-homocysteine has the following formula:

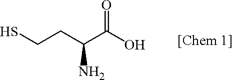

L-homocysteine sulfide has the following formula:

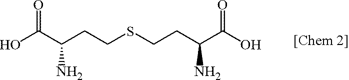 [SMILES: N[C@@H](CCSCC[C@H](N)C(=O)O)C(=O)O]

L-homocystine has the following formula:

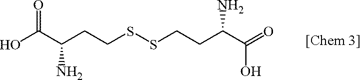 [SMILES: N[C@@H](CCSSCC[C@H](N)C(=O)O)C(=O)O]

In addition, it has been observed that the configuration of the asymmetric carbon atoms is retained throughout the reaction. Therefore, the functionalized mercaptan of formula (I) obtained according to the process of the invention may be enantiomerically pure.

The process according to the invention is also easy to implement industrially. It can be carried out in solution under mild temperature and pressure conditions. The use of salts makes it possible advantageously to avoid operators handling hydrogen sulfide, which is a toxic gas.

The yield obtained can be greater than or equal to 85%, preferably greater than or equal to 90%, for example between 90% and 100%, limits included. Surprisingly, the process according to the invention makes it possible in particular to obtain a yield of 100%, i.e. an increase of close to 20% compared to other processes.

The present invention thus relates to a process for synthesizing at least one functionalized mercaptan of the following general formula (I):

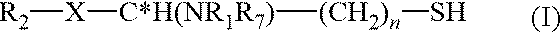

in which,
R₁ and R₇, which are identical or different, are a hydrogen atom or an aromatic or nonaromatic, linear, branched or cyclic, saturated or unsaturated, hydrocarbon chain of 1 to 20 carbon atoms which may comprise one or more heteroatoms;
X is chosen from —C(═O)—, —CH₂— or —CN;
R₂ is:
   (i) either absent when X represents —CN,
   (ii) or a hydrogen atom,
   (iii) or —OR₃, R₃ being a hydrogen atom or an aromatic or nonaromatic, linear, branched or cyclic, saturated or unsaturated, hydrocarbon chain of 1 to 20 carbon atoms which may comprise one or more heteroatoms,
   (iv) or —NR₄R₅, R₄ and R₅, which are identical or different, being a hydrogen atom or an aromatic or nonaromatic, linear, branched or cyclic, saturated or unsaturated, hydrocarbon chain of 1 to 20 carbon atoms which may comprise one or more heteroatoms;
n is equal to 1 or 2; and * represents an asymmetric carbon;
said process comprising the steps of:
a) provision of at least one compound of the following general formula (II):

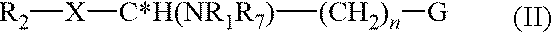

in which *, R₁, R₂, R₇, X and n are as defined for formula (I) and
G represents either (i) R₆-C(O)-O-, or (ii) (R₇O)(R₈O)-P(O)-O-, or (iii) R₉O-SO₂-O-; with
R₆ being a hydrogen atom or a linear, branched or cyclic, saturated or unsaturated hydrocarbon chain of 1 to 20 carbon atoms which may comprise one or more aromatic groups and may be substituted by one or more groups chosen from -OR₁₀, (=O), -C(O)OR₁₁, -NR₁₂R₁₃;
R₁₀, R₁₁, R₁₂ and R₁₃ being independently chosen from:
   H or a linear, branched or cyclic, saturated or unsaturated hydrocarbon chain of 1 to 20 carbon atoms;
R₇ and R₈, which are identical or different, being a proton, an alkali metal, an alkaline earth metal or an ammonium;
R₉ is chosen from a proton, an alkali metal, an alkaline earth metal or an ammonium;
b) provision of at least one hydrosulfide salt and/or sulfide salt or H₂S;
c) reaction between said at least one compound of formula (II) and said at least one hydrosulfide and/or sulfide salt or H₂S in the presence of at least one enzyme chosen from sulfhydrylases, and preferably a sulfhydrylase associated with said compound of formula (II); said reaction being performed essentially in the absence of oxygen, preferably in the absence of oxygen;
d) obtaining of at least one functionalized mercaptan of formula (I);
e) optional separation of said at least one functionalized mercaptan of formula (I) which is obtained in step d); and
f) optional additional functionalization and/or optional deprotection of the functionalized mercaptan of formula (I) which is obtained in step d) or e); and
wherein steps a) and b) are optionally performed simultaneously.

Oxygen is understood to mean, in particular, dioxygen O₂.

Step c) is thus carried out essentially in the absence of oxygen, or even in the absence of oxygen. More particularly, “essentially in the absence of oxygen” is understood to mean that an amount of oxygen may remain in the reaction mixture and/or in the gas phase (contained in the gas headspace of the reactor) such that the amount of sulfides and/or polysulfides produced is less than or equal to 5% by weight relative to the total weight of the compound of formula (I) produced. Preferentially, “essentially in the absence of oxygen” is understood to mean that the reaction mixture contains less than 0.0015% oxygen (preferably strictly less than 0.0015%) by weight relative to the total weight of the reaction mixture and/or that the gas phase (contained in the gas headspace of the reactor) contains less than 21% oxygen (preferably strictly less than 21%) by volume relative to the total volume of said gas phase.

Thus, step c) can alternatively be as follows:

c) reaction between said at least one compound of formula (II) and said at least one hydrosulfide and/or sulfide salt or H₂S in the presence of at least one enzyme chosen from sulfhydrylases, and preferably a sulfhydrylase associated with said compound of formula (II); said reaction being performed in a reactor in which the reaction mixture comprises between 0 and 0.0015% oxygen (preferably strictly less than 0.0015%) by weight relative to the total weight of the reaction mixture and/or the gas phase contained in the gas headspace of the reactor comprises between 0 and 21% oxygen (preferably strictly less than 21%) by volume relative to the total volume of the gas phase.

In particular, the amount of oxygen in the reaction mixture and/or in the gas phase (contained in the gas headspace) is such that the amount of sulfides and/or polysulfides produced is less than or equal to 5% by weight relative to the total weight of the compound of formula (I) produced.

For example, step c) can be performed in a closed reactor (i.e. without supply of oxygen from the air). Highly preferably, the gas phase (contained in the gas headspace) does not comprise oxygen, in particular when H₂S is used. Preferably, the gas phase (contained in the gas headspace) does not comprise oxygen and the reaction mixture comprises between 0 and 0.0015% oxygen (preferably strictly less than 0.0015%) by weight relative to the total weight of the reaction mixture.

Indeed, the O₂/H₂S mixture can present an explosion risk, which obviously involves a risk to the safety of the operators.

In particular, “gas headspace” is understood to mean the space in the reactor located above the reaction mixture, preferably above the liquid reaction mixture. More particularly, “gas headspace” is understood to mean the space located between the surface of the liquid reaction mixture and the top of the reactor (i.e. the upper part of the reactor comprising the gas phase when the lower part of the reactor comprises a liquid phase). The gas headspace in particular comprises a gas phase.

The reactants are in particular introduced into the reactor in amounts such that a gas headspace is located above the reaction mixture contained in the reactor.

In particular, it is understood that when H₂S is used, a part of the H₂S is dissolved in the reaction mixture so that the reaction of step c) is performed while the other part is located in gas form in the gas headspace of the reactor.

More particularly, said at least one compound of formula (II), said at least one hydrosulfide salt and/or sulfide salt or H₂S, and said at least one sulfhydrylase form a reaction mixture (or medium). Said reaction mixture may thus comprise:
at least one compound of formula (II) as defined below,
at least one hydrosulfide and/or sulfide salt as defined below or H₂S,
at least one sulfhydrylase as defined below,
optionally its cofactor as defined below,
optionally a base as defined below, and
optionally a solvent, preferably water.

Said reaction mixture can be prepared by adding said compound of formula (II), said hydrosulfide and/or sulfide salt or H₂S and said sulfhydrylase in any order.

For example, it is possible to first mix said compound of formula (II) with said salt or H₂S, then to add the sulfhydrylase, optionally with its cofactor, to start said reaction of step c).

Notably, it is the addition of the third component, irrespective of what it is, in particular the sulfhydrylase, which enables the reaction to start.

Preferably, the compound of formula (II) is in the form of a solution, more preferentially in the form of an aqueous solution.

Preferably, when hydrosulfide and/or sulfide salts are used, these are used in the form of a solution and more preferentially in the form of an aqueous solution.

When H₂S is used, it is generally in gaseous form. It may in particular be introduced into the reaction mixture by bubbling. The bubbling can be effected by mixing H₂S with an inert gas, for example dinitrogen, argon or methane, preferably dinitrogen. H₂S may thus be present in dissolved form in the reaction mixture.

Conventional methods can be used for performing step c) essentially in the absence of oxygen, or even in the absence of oxygen.

According to one embodiment, prior to step c) the oxygen is removed from the reaction mixture, for example by degassing.

According to another embodiment, prior to step c) the oxygen is removed separately from each of the components or from the mixture of at least two thereof that are going to form the reaction mixture. For example, each of the solutions comprising the compound of formula (II), the hydrosulfide and/or sulfide salt where this is used, the sulfhydrylase and optionally the solvent, are degassed.

It is also possible to remove the oxygen from the headspace of the reactor in which step c) takes place, preferably by degassing.

The reactor can also be inertized with an inert gas such as dinitrogen, argon or methane, preferably dinitrogen.

When H₂S is used, this being gaseous, degassing is of course not carried out for this reactant.

H₂S generally does not comprise oxygen.

Various techniques may also be combined with each other.

Preferably, the absence of oxygen is achieved in the following way:
the reactor is inertized with an inert gas such as dinitrogen, argon or methane, preferably dinitrogen; and
each of the solutions comprising the compound of formula (II), the hydrosulfide and/or sulfide salt where this is used, the sulfhydrylase and optionally the solvent, are degassed.

Industrial degassing methods are well known and mention may for example be made of the following:
pressure reduction (vacuum degassing),
thermal regulation (increasing the temperature for an aqueous solvent and lowering the temperature for an organic solvent),
membrane degassing,
degassing by alternating freeze-pump-thaw cycles,
degassing by sparging with an inert gas (for example argon, dinitrogen or methane).

According to one embodiment, in step c) the oxygen is neither present in a form dissolved in a liquid (in particular in the reaction mixture) nor in gaseous form (in particular in the headspace of the reactor in which step c) is taking place).

Preferably, the hydrosulfide salt and/or the sulfide salt or the H₂S is in excess, preferably in molar excess, relative to the compound of formula (II), preferably during step c) and more preferentially during the entire duration of step c).

The hydrosulfide and/or sulfide salt or the H₂S can therefore be in a superstoichiometric amount relative to the amount of the compound of formula (II), preferably during step c) and more preferentially during the entire duration of step c).

In particular, the molar ratio [hydrosulfide salt and/or sulfide salt] / [compound of formula (II)] or the molar ratio H₂S / compound of formula (II) is comprised between 1.5 and 10, preferentially between 2 and 8, for example between 3.5 and 8, and even more preferentially between 3.5 and 5, limits included, preferably during step c) and more preferentially during the entire duration of step c). Said ratio may be kept constant during the entire duration of step c).

Step c) can be carried out in solution, in particular in aqueous solution. For example, the solution comprises between 50% and 99% by weight of water, preferably between 75% and 97% by weight of water, relative to the total weight of the solution, limits included.

The pH of the reaction mixture in step c) can be between 4 and 9, for example between 5 and 8, preferably between 6 and 7.5, and more particularly between 6.2 and 7.2, limits included, in particular when the reaction mixture is an aqueous solution.

The pH can in particular be adjusted within the abovementioned ranges according to the operating optimum of the chosen sulfhydrylase. The pH can be determined by conventionally known methods, for example with a pH probe.

According to a preferred embodiment, step c) can be carried out in accordance with the following two steps c1) and c2):
c1) reaction between said at least one compound of formula (II) and said at least one hydrosulfide and/or sulfide salt or H₂S in the presence of at least one enzyme chosen from sulfhydrylases, and preferably a sulfhydrylase associated with said compound of formula (II); said reaction being performed essentially in the absence of oxygen, preferably in the absence of oxygen, and in solution;
c2) adjustment of the pH of said solution by addition of a base so as to obtain a pH of between 4 and 9, for example between 5 and 8, preferably between 6 and 7.5, and more particularly between 6.2 and 7.2, limits included.

Any type of base may be used in step c2), preferably a base comprising a sulfur atom. A base is understood in particular to be a compound or a mixture of compounds having a pH of greater than 7, preferably between 8 and 14, limits included. The base can be chosen from hydrosulfide salts and/or sulfide salts as defined below, sodium hydroxide, potassium hydroxide or ammonia. The base can in particular be chosen from hydrosulfide salts and/or sulfide salts as defined below. Preferably, said base is the hydrosulfide salt and/or the sulfide salt used in step c1). The preferred base is ammonium hydrosulfide (NH₄SH).

The base can be added at a concentration of between 0.1 and 10 M, preferably between 0.5 and 10 M, more preferably between 0.5 and 5 M, limits included. Use will in particular be made of concentrated bases so as to limit the dilution of the reaction mixture when adding the base.

The temperature during step c) can be between 10° C. and 60° C., preferably between 20° C. and 40° C. and more particularly between 25° C. and 40° C., limits included. The pressure during step c) is generally atmospheric pressure. Step c) may be performed batchwise, semi-continuously or continuously. Any type of reactor may be suitable.

The separation step e) can be performed according to any technique known to a person skilled in the art. In particular, when the final product is a solid:
by extraction and/or decantation with a solvent which is immiscible in the reaction medium, followed by an evaporation of said solvent;
by precipitation (by partial evaporation of the solvents or by addition of a solvent in which the compound of interest is less soluble). This precipitation is generally followed by a step of filtration according to any method known to a person skilled in the art. The final product can then be dried; or
by selective precipitation via adjustment of the pH as a function of the respective solubilities of the different compounds.

Homocysteine may in particular be recovered in solid form.

When the final product is in liquid form, the separation can be performed by distillation or by distillation or evaporation preceded by a liquid/liquid extraction.

Step f) of additional functionalization and/or optional deprotection can make it possible to obtain additional chemical functions and/or to deprotect certain chemical functions by conventional methods. For example, if X-R₂ represents a carboxyl functional group, the latter can be esterified, reduced to an aldehyde, reduced to an alcohol and then esterified, amidated, nitrilated or others. All the functional groups can be obtained and/or deprotected by a person skilled in the art depending on the final use which is intended for said functionalized mercaptan of formula (I).

Thus, the functionalized mercaptan of formula (I) obtained on conclusion of step d) or e) may be subjected to one or more additional chemical reactions in order to obtain one or more mercaptan derivatives with different functionalities, said chemical reactions being reactions that are well known to a person skilled in the art.

The expression “between X and X” includes the limits mentioned, unless specified otherwise.

A heteroatom is understood in particular to be an atom chosen from O, N, S, P and halogens.

An unsaturated hydrocarbon chain is understood to be a hydrocarbon chain comprising at least one double or triple bond between two carbon atoms.

### Functionalized Mercaptans of General Formula (I)

The process according to the invention is targeted at obtaining functionalized mercaptans of the following general formula (I):

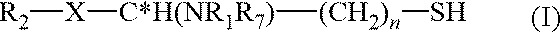

in which,
R₁ and R₇, which are identical or different, are a hydrogen atom or an aromatic or nonaromatic, linear, branched or cyclic, saturated or unsaturated, hydrocarbon chain of 1 to 20 carbon atoms which may comprise one or more heteroatoms;
X is chosen from -C(=O)-, -CH₂- or -CN;
R₂ is:
   (i) either absent when X represents -CN,
   (ii) or a hydrogen atom,
   (iii) or -OR₃, R₃ being a hydrogen atom or an aromatic or nonaromatic, linear, branched or cyclic, saturated or unsaturated, hydrocarbon chain of 1 to 20 carbon atoms which may comprise one or more heteroatoms,
   (iv) or -NR₄R₅, R₄ and R₅, which are identical or different, being a hydrogen atom or an aromatic or nonaromatic, linear, branched or cyclic, saturated or unsaturated, hydrocarbon chain of 1 to 20 carbon atoms which may comprise one or more heteroatoms;
n is equal to 1 or 2; and * represents an asymmetric carbon.

These mercaptans are referred to as functionalized because, in addition to the chemical function -SH, they also comprise at least one amine-type function -NR₁R₇.

Preferably, n is equal to 2.

Preferably, X is -C(=O)-.

Preferably, R₂ is -OR₃ with R₃ as defined above. R₃ may in particular be a hydrogen atom or a linear or branched, saturated hydrocarbon chain of 1 to 10 carbon atoms, preferably of 1 to 5 carbon atoms. In particular, R₃ is H.

R₁ and R₇, which are identical or different, are preferably a hydrogen atom or a linear or branched, saturated hydrocarbon chain of 1 to 10 carbon atoms, preferably of 1 to 5 carbon atoms. Preferably, R₁ and R₇ are H.

In particular, X is -C(=O)- and R₂ is -OR₃ with R₃ as defined above.

The functionalized mercaptans of formula (I) may be chosen from the group consisting of homocysteine, cysteine, and derivatives of these.

In particular, the functionalized mercaptans of formula (I) are L-homocysteine and L-cysteine.

A preferred functionalized mercaptan of formula (I) is homocysteine, and very particularly L-homocysteine of the following formula:

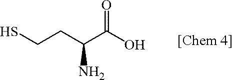

For L-homocysteine, n is equal to 2, X is -C(=O)-, R₂ is -OR₃ with R₃ being H and R₁ and R₇ are H.

The functionalized mercaptans of formula (I) are chiral compounds. They may be obtained in enantiomerically pure form by the process according to the invention. In the present description, when the enantiomeric form is not specified, the compound is included whatever its enantiomeric form.

According to one embodiment, the reaction mixture at the end of step c) does not comprise sulfide or polysulfide and in particular does not comprise sulfide or polysulfide corresponding to the functionalized mercaptan of formula (I) obtained. For example, the reaction mixture at the end of step c) comprises less than 10 mol%, preferably less than 5 mol%, of sulfides and polysulfides relative to the total number of moles of compound of formula (II) converted into compound of formula (I).

Sulfide is understood in particular to be the sulfide corresponding to the compound of formula (I) which is that of the following formula (III):
R₂-X-C*H(NR₁R₇)-(CH₂)ₙ-S-(CH₂)ₙ-(NR₁R₇)C*H-X-R₂ (III)
with *, R₁, R₂, R₇, X and n as defined above.

Polysulfide is understood in particular to be the polysulfide corresponding to the compound of formula (I) which is that of the following formula (IV):
R₂-X-C*H(NR₁R₇)-(CH₂)ₙ-(S)ₘ-(CH₂)ₙ-(NR₁R₇)C*H-X-R₂ (IV)
with *, R₁, R₂, R₇, X and n as defined above and m being an integer between 2 and 6, limits included, for example m is equal to 2 or 3.

Preferably, m is equal to 2 (which corresponds to a disulfide).

In particular, the reaction mixture at the end of step c) does not comprise L-homocysteine sulfide or L-homocystine when the compound of formula (I) is L-homocysteine.

Preferably, following the reaction of the compound of formula (II) with said at least one hydrosulfide and/or sulfide salt or H₂S during step c), there are obtained a functionalized mercaptan of formula (I) as defined below and a compound of formula (V) GH, where G is as defined above, that is to say, a compound of the type: (i′) R₆-C(O)-OH, (ii′) (R₇O)(R₈O)-P(O)-OH, or (iii′) R₉O-SO₂-OH; with R₆, R₇, R₈ and R₉ being as defined below. In particular, when the compound (II) is O-acetyl-L-homoserine, L-homocysteine and acetic acid are obtained. The compounds of formula (V) may be responsible for the acidification of the reaction mixture during step c). Thus, it is possible to maintain the pH of the reaction mixture between 4 and 9, for example between 5 and 8, preferably between 6 and 7.5, and more particularly between 6.2 and 7.2, in particular during step c) as mentioned above and in particular by addition of a base as defined above.

### Hydrosulfide Salt and/or Sulfide Salt or H₂S

The present invention can be carried out in the presence of a hydrosulfide and/or sulfide salt or in the presence of H₂S (hydrogen sulfide).

Said salt is generally provided in the form of a solution, preferably an aqueous solution.

Said at least one hydrosulfide and/or sulfide salt can be chosen from the group consisting of: ammonium hydrosulfide, alkali metal hydrosulfides, alkaline earth metal hydrosulfides, alkali metal sulfides and alkaline earth metal sulfides.

Alkali metals are understood to be lithium, sodium, potassium, rubidium and caesium, preferably sodium and potassium.

Alkaline earth metals are understood to be beryllium, magnesium, calcium, strontium and barium, preferably calcium.

In particular, said at least one hydrosulfide salt and/or sulfide salt can be chosen from the group consisting of:

ammonium hydrosulfide NH₄SH, sodium hydrosulfide NaSH, potassium hydrosulfide KSH, calcium hydrosulfide Ca(SH)₂, sodium sulfide Na₂S, ammonium sulfide (NH₄)₂S, potassium sulfide K₂S and calcium sulfide CaS. The preferred hydrosulfide is ammonium hydrosulfide NH₄SH. The ammonium released during the reaction may for example be reused as a nitrogen source for the growth of microorganisms, in particular microorganisms expressing or overexpressing sulfhydrylase. For example, the microorganisms may be chosen from the group consisting of: cells of bacteria such as Escherichia coli, Bacillus sp., or Pseudomonas, cells of yeast such as Saccharomyces cerevisiae or Pichia pastoris, cells of fungi such as Aspergillus niger, Penicillium funiculosum or Trichoderma reesei, insect cells such as Sf9 cells, or else mammalian (in particular human) cells such as the HEK 293, PER-C6 or CHO cell lines.

More particularly, use will be made of bacterial cells and even more preferentially of E. coli cells.

### Compounds of General Formula (II)

For the compounds of the following general formula (II):

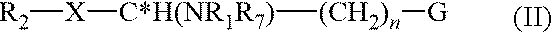

*, R₁, R₂, R₇, X and n are as defined above for the compounds of formula (I), and
G represents either (i) R₆-C(O)-O-, or (ii) (R₇O)(R₈O)-P(O)-O-, or (iii) R₉O-SO₂-O-;
with R₆ being a hydrogen atom or a linear, branched or cyclic, saturated or unsaturated hydrocarbon chain of 1 to 20, preferably 1 to 10, carbon atoms which may comprise one or more aromatic groups and may be substituted by one or more groups chosen from -OR₁₀, (=O), -C(O)OR₁₁, and -NR₁₂R₁₃;
R₁₀, R₁₁, R₁₂ and R₁₃ being independently chosen from:
   H or a linear, branched or cyclic, saturated or unsaturated hydrocarbon chain of 1 to 20, preferably 1 to 10, carbon atoms;
R₇ and R₈, which are identical or different, being a proton, an alkali metal, an alkaline earth metal or an ammonium, preferably a proton or an alkali metal and more particularly H⁺ or Na⁺;
R₉ is chosen from a proton, an alkali metal, an alkaline earth metal or an ammonium, preferably a proton or an alkali metal and more particularly a proton H⁺ or Na⁺;
In particular, G represents either R₆-C(O)-O- or R₉O-SO₂-O-; preferably G is R₆-C(O)-O-.

In particular, R₆ is a hydrogen atom or a linear or branched, saturated or unsaturated hydrocarbon chain of 1 to 10, preferably 1 to 5, carbon atoms which may be substituted by one or more groups chosen from -OR₁₀, (=O) and -C(O)OR₁₁; R₁₀ and R₁₁ being independently chosen from:

H or a linear or branched, saturated or unsaturated hydrocarbon chain of 1 to 10, preferably 1 to 5, carbon atoms.

More particularly, R₁₀ and R₁₁ are H. In particular, R₁₂ and R₁₃ are H.

Aromatic group is understood preferentially to be the phenyl group.

The compound of general formula (II) is in particular a derivative of serine (when n is equal to 1) or homoserine (when n is equal to 2), in particular of L-serine or of L-homoserine. It may for example be chosen from the group consisting of:

O-phospho-L-homoserine, O-succinyl-L-homoserine, O-acetyl-L-homoserine, O-acetoacetyl-L-homoserine, O-propio-L-homoserine, O-coumaroyl-L-homoserine, O-malonyl-L-homoserine, O-hydroxymethylglutaryl-L-homoserine, O-pimelyl-L-homoserine, O-sulfato-L-homoserine, O-phospho-L serine, O-succinyl-L-serine, O-acetyl-L-serine, O-acetoacetyl-L-serine, O-propio-L-serine, O-coumaroyl-L-serine, O-malonyl-L-serine, O-hydroxymethylglutaryl-L-serine, O-pimelyl-L-serine and O-sulfato-L-serine.

More particularly, it may be chosen from the group consisting of:

O-phospho-L-homoserine, O-succinyl-L-homoserine, O-acetyl-L-homoserine, O-acetoacetyl-L-homoserine, O-propio-L-homoserine, O-coumaroyl-L-homoserine, O-malonyl-L-homoserine, O-hydroxymethylglutaryl-L-homoserine, O-pimelyl-L-homoserine and O-sulfato-L-homoserine.

The compound of general formula (II) may be chosen from the group consisting of: O-phospho-L-homoserine, O-succinyl-L-homoserine, O-acetyl-L-homoserine, O-sulfato-L-homoserine and O-propio-L-homoserine.

The compound of general formula (II) may be chosen from the group consisting of: O-phospho-L-homoserine, O-succinyl-L-homoserine, O-acetyl-L-homoserine.

The compound of formula (II) which is very particularly preferred is O-acetyl-L-homoserine (OAHS), a compound for which n is equal to 2, X is -C(=O)-, R₂ is -OR₃ with R₃ being H, R₁ and R₇ are H and G is -O-C(O)-R₆ with R₆ being a methyl.

The compounds of formula (II) are either commercially available or obtained via any technique known to a person skilled in the art.

They may be obtained by a fermentation process from a source of hydrocarbon and nitrogen, for example as described in the application WO 2008/013432.

They may be obtained, for example, by fermentation of a renewable starting material. The renewable starting material may be chosen from glucose, sucrose, starch, molasses, glycerol and bioethanol, preferably glucose.

The L-serine derivatives may also be produced from the acetylation of L-serine, the L-serine itself possibly being obtained by fermentation of a renewable starting material. The renewable starting material may be chosen from glucose, sucrose, starch, molasses, glycerol and bioethanol, preferably glucose.

The L-homoserine derivatives may also be produced from the acetylation of L-homoserine, the L-homoserine itself possibly being obtained by fermentation of a renewable starting material. The renewable starting material may be chosen from glucose, sucrose, starch, molasses, glycerol and bioethanol, preferably glucose.

### Sulfhydrylases

The reaction between said at least one compound of formula (II) and said at least one hydrosulfide and/or sulfide salt as defined above or H₂S is performed in the presence of at least one enzyme chosen from sulfhydrylases, preferably a sulfhydrylase associated with said compound of formula (II). The sulfhydrylase associated with a compound of formula (II) is easily identifiable since it shares the same name, for example O-acetyl-L-homoserine sulfhydrylase (OAHS Sulfhydrylase) is associated with O-acetyl-L-homoserine.

The sulfhydrylase in particular enables catalysis of the reaction between said compound of formula (II) and said salt or H₂S. “Catalyst” is understood generally to be a substance which accelerates a reaction and which is unchanged at the end of this reaction. The sulfhydrylase, and optionally its cofactor, can be used in a catalytic amount. “Catalytic amount” is understood in particular to be an amount sufficient to catalyse a reaction. More particularly, a reagent used in a catalytic amount is used in a smaller amount, for example between around 0.01% and 20% by weight, limits included, relative to the amount by weight of a reagent used in stoichiometric proportion.

Said sulfhydrylase enzyme preferably belongs to the transferases class, notably designated by the EC 2.X.X.XX (or noted EC 2) classification. The EC classification for « Enzyme Commission numbers » is widely used and can be found on the website https://enzyme.expasy.org/. In particular, said enzyme is chosen among sulfhydrylases of the EC 2.5.X.XX class (or noted EC 2.5.), meaning transferases transferring alkyl or aryl group, other than methyl group.

The sulfhydrylases are in particular of the class EC 2.5.1.XX (with XX varying depending on the substrate of the enzyme).

For example:
O-acetylhomoserine sulfhydrylase is of type EC 2.5.1.49.
O-phosphoserine sulfhydrylase is of type EC 2.5.1.65.
O-succinylhomoserine sulfhydrylase is of type EC 2.5.1.49.

For example :
O-acetyl-L-homoserine sulfhydrylase is of type EC 2.5.1.49.
O-phospho-L-serine sulfhydrylase is of type EC 2.5.1.65.
O-succinyl-L-homoserine sulfhydrylase is of type EC 2.5.1.49.

Thus, in particular when the compound of formula (II) is a derivative of L-homoserine or of L-serine, the sulfhydrylase used can be chosen from O-phospho-L-homoserine sulfhydrylase, O-succinyl-L-homoserine sulfhydrylase, O-acetyl-L-homoserine sulfhydrylase, O-acetoacetyl-L-homoserine sulfhydrylase, O-propio-L-homoserine sulfhydrylase, O-coumaroyl-L-homoserine sulfhydrylase, O-malonyl-L-homoserine sulfhydrylase, O-hydroxymethylglutaryl-L-homoserine sulfhydrylase, O-pimelyl-L-homoserine sulfhydrylase, O-sulfato-L-homoserine sulfhydrylase, O-phospho-L-serine sulfhydrylase, O-succinyl-L-serine sulfhydrylase, O-acetyl-L-serine sulfhydrylase, O-acetoacetyl-L-serine sulfhydrylase, O-propio-L-serine sulfhydrylase, O-coumaroyl-L-serine sulfhydrylase, O-malonyl-L-serine sulfhydrylase, O-hydroxymethylglutaryl-L-serine sulfhydrylase, O-pimelyl-L-serine sulfhydrylase and O-sulfato-serine sulfhydrylase.

More particularly, the sulfhydrylase used can be chosen from O-phospho-L-homoserine sulfhydrylase, O-succinyl-L-homoserine sulfhydrylase, O-acetyl-L-homoserine sulfhydrylase, O-acetoacetyl-L-homoserine sulfhydrylase, O-propio-L-homoserine sulfhydrylase, O-coumaroyl-L-homoserine sulfhydrylase, O-malonyl-L-homoserine sulfhydrylase, O-hydroxymethylglutaryl-L-homoserine sulfhydrylase, O-pimelyl-L-homoserine sulfhydrylase, O-sulfato-L-homoserine sulfhydrylase.

In particular, the sulfhydrylase can be chosen from O-phospho-L-homoserine sulfhydrylase, O-succinyl-L-homoserine sulfhydrylase, O-acetyl-L-homoserine sulfhydrylase, O-sulfato-L-homoserine sulfhydrylase and O-propio-L-homoserine sulfhydrylase.

The sulfhydrylase can be chosen from O-phospho-L-homoserine sulfhydrylase, O-succinyl-L-homoserine sulfhydrylase and O-acetyl-L-homoserine sulfhydrylase.

Very particularly preferably, the enzyme is O-acetyl-L-homoserine sulfhydrylase (OAHS Sulfhydrylase).

Said sulfhydrylase, and in particular the O-acetyl-L-homoserine sulfhydrylase, may originate from or be derived from the following bacterial strains: Pseudomonas sp., Chromobacterium sp., Leptospira sp. ou Hyphomonas sp..

The sulfhydrylases can function, as is perfectly known to a person skilled in the art, in the presence of a cofactor such as pyridoxal 5′-phosphate (also known as PLP) or one of its analogues, preferably pyridoxal 5′-phosphate.

Among the analogues of the cofactor pyridoxal phosphate, mention may be made of α⁵-pyridoxalmethylphosphate, 5′-methylpyridoxal-P, pyridoxal 5′-sulfate, α⁵-pyridoxalacetic acid or any other known derivative (Groman et al., Proc. Nat. Acad. Sci. USA Vol. 69, No. 11, pp. 3297-3300, November 1972).

According to one embodiment, a cofactor of the sulfhydrylase can be added to the reaction mixture. Thus, a cofactor of the sulfhydrylase, for example pyridoxal 5′-phosphate, may be provided prior to step c), or may be added during step c). When step c) is performed in aqueous solution, the enzyme and optionally its cofactor can be dissolved beforehand in water before being added to said solution.

According to another embodiment, cells, for example bacterial cells or other cells, may produce or even overproduce said cofactor while simultaneously expressing or overexpressing the sulfhydrylase enzyme, so as to avoid a step of supplementing said cofactor.

According to one embodiment, the sulfhydrylase, and optionally its cofactor, are:
either in isolated and/or purified form, for example in aqueous solution;

The isolation and/or the purification of said produced enzyme can be carried out by any means known to a person skilled in the art. It may for example involve a technique chosen from electrophoresis, molecular sieving, ultracentrifugation, differential precipitation, for example with ammonium sulfate, ultrafiltration, membrane or gel filtration, ion exchange, separation via hydrophobic interactions, or affinity chromatography, for example of IMAC type.
or present in a crude extract, that is to say in an extract of milled cells (lysate); the enzyme of interest may or may not be overexpressed in said cells, hereinafter denoted host cells. The host cell may be any host cell appropriate for the production of the enzyme of interest from the expression of the corresponding coding gene. This gene will then be either located in the genome of the host or carried by an expression vector.

For the purposes of the present invention, “host cell” is in particular understood to be a prokaryotic or eukaryotic cell. Host cells commonly used for the expression of recombinant or non-recombinant proteins include in particular cells of bacteria such as Escherichia coli or Bacillus sp., or Pseudomonas, cells of yeast such as Saccharomyces cerevisiae or Pichia pastoris, cells of fungi such as Aspergillus niger, Penicillium funiculosum or Trichoderma reesei, insect cells such as Sf9 cells, or else mammal (in particular human) cells such as the HEK 293, PER-C6 or CHO cell lines.

Preferably, the enzyme of interest and optionally the cofactor are expressed in the bacterium Escherichia coli. Preferentially, the enzyme of interest is expressed within a strain of Escherichia coli such as for example Escherichia coli BL21 (DE3).

The cell lysate can be obtained according to various known techniques such as sonication, pressure (French press), via the use of chemical agents (e.g. xylene, triton), etc. The lysate obtained corresponds to a crude extract of milled cells.
or present in whole cells. For this, the same techniques as above can be used, without performing the cell lysis step.

According to one embodiment, the amount of biomass expressing the sulfhydrylase enzyme, relative to the mass of the compound of formula (II), is between 0.1% and 10% by weight, preferably between 1% and 5% by weight, and/or the amount of cofactor relative to the compound of formula (II) is between 0.1% and 10% by weight, preferably between 0.5% and 5% by weight, limits included.

The reaction mixture may also comprise:
optionally one or more solvents chosen from water, buffers such as phosphate buffers, Tris-HCl, Tris base, ammonium bicarbonate, ammonium acetate, HEPES (4-(2-hydroxyethyl)-1-piperazineethanesulfonic acid), CHES (N-cyclohexyl-2-aminoethanesulfonic acid), or salts such as sodium chloride, potassium chloride, or mixtures thereof;
optionally additives such as surfactants, in order in particular to promote the solubility of one or more reagents or substrates.

The various components which can be used for the reaction of step c) above are readily commercially obtainable or can be prepared according to techniques well known to a person skilled in the art. These different elements may be in solid, liquid or gaseous form and may very advantageously be rendered into solution or dissolved in water or any other solvent to be used in the process of the invention. The enzymes used may also be grafted onto a support (in the case of supported enzymes).

According to a preferred embodiment, said compound of formula (II) is O-acetyl-L-homoserine, the enzyme used is O-acetyl-L-homoserine sulfhydrylase and the functionalized mercaptan of formula (I) obtained is L-homocysteine.

According to a preferred embodiment, said compound of formula (II) is O-acetyl-L-homoserine, the salt is ammonium hydrosulfide, the enzyme used is O-acetyl-L-homoserine sulfhydrylase and the functionalized mercaptan of formula (I) obtained is L-homocysteine.

The present invention also relates to a composition, preferably an aqueous solution, comprising:
a compound of formula (II) as defined above;
a sulfhydrylase, preferably a sulfhydrylase associated with the compound of formula (II), said sulfhydrylase being as defined above; and
a hydrosulfide salt and/or a sulfide salt as defined above or H₂S in excess, preferably NH₄SH in excess.

Preferably, said composition comprises:
O-acetyl-L-homoserine;
O-acetyl-L-homoserine sulfhydrylase; and
NH₄SH or H₂S in excess.

Said composition in particular corresponds to the reaction mixture as defined above.

The conditions, characteristics and optional additional components are the same as those defined for the reaction mixture as defined above.

In particular, the composition according to the invention does not comprise dissolved oxygen. Preferably, the hydrosulfide and/or sulfide salt or the H₂S is in excess, preferably in molar excess, relative to the compound of formula (II). The hydrosulfide and/or sulfide salt or the H₂S can therefore be in a superstoichiometric amount relative to the amount of the compound of formula (II).

In particular, the molar ratio [hydrosulfide salt and/or sulfide salt] / [compound of formula (II)] or H₂S / compound of formula (II) is between 1.5 and 10, preferentially between 2 and 8, for example between 3.5 and 8, and even more preferentially between 3.5 and 5, limits included.

The composition may also comprise a cofactor of the sulfhydrylase as defined above.

In particular, the composition according to the invention makes it possible to implement the process according to the invention.

### EXAMPLES

The examples which follow make it possible to illustrate the present invention but are not under any circumstances limiting.

The usual definitions of conversion, of selectivity and of yield are as follows:
Conversion = (number of moles of reactant in the initial state - number of moles of reactant remaining after the reaction) / (number of moles of reactant in the initial state)
Selectivity = Number of moles of reactant converted into the desired product / (number of moles of reactant in the initial state - number of moles of reactant remaining after the reaction)
Yield = conversion X selectivity

### Example 1: Comparative Process for Synthesizing L-Homocysteine in the Presence of Oxygen and in the Presence of a Stoichiometric Amount of NaSH relative to OAHS.

### Step 1.

O-Acetyl-L-homoserine was synthesized from L-homoserine and acetic anhydride according to the protocol described in the works of Sadamu Nagai, “Synthesis of O-acetyl-L-homoserine”, Academic Press (1971), vol. 17, p. 423-424.

### Step 2.

5.25 g/l of O-acetyl-L-homoserine originating from step 1), this product being dissolved in 140 ml of water, are introduced into a thermostatically controlled 250 ml glass reactor. The solution is brought to 37° C. with mechanical stirring. Next, a stoichiometric amount of NaSH dihydrate is added to the reactor (i.e. 3 g/l). The pH of the reaction medium is adjusted to a value of 6.5 using an aqueous ammonia solution (4 M) and then 5 g/l of OAHS Sulfhydrylase and 0.4 g/l of pyridoxal phosphate cofactor are added to the reaction mixture. The pH is maintained at a setpoint value of 6.5 using an aqueous ammonia solution (4 M).

The analyses by potentiometry, HPLC and NMR show a gradual disappearance of the reagents (OAHS and NaSH) and the gradual appearance of several products over time. The compounds thus formed are predominantly:
L-homocysteine,
L-homocysteine sulfide (4,4′-sulfanediylbis(2-aminobutanoic acid) / L-homolanthionine), and
L-homocystine (disulfide / L-4,4′-dithiobis(2-aminobutanoic acid)).

An analysis of the reaction medium at the end time made it possible to show that all of the OAHS is consumed at the end of the reaction since it is not detectable even in the form of traces.

The molar selectivities obtained with respect to the transformed OAHS (i.e. expressed in mol% of the different compounds present in the final mixture excluding water, acetic acid and PLP cofactor) are as follows:
- 31% of L-homocysteine and
- 69% of homocysteine sulfide (L-homolanthionine / 4,4′-sulfanediylbis(2-aminobutanoic acid)) and homocystine (disulfide / L-4,4′-dithiobis(2-aminobutanoic acid)).

The molar yield of L-homocysteine is 31%.

### Example 2: Comparative Process for Synthesizing L-Homocysteine in the Presence of Oxygen and in the Presence of a Superstoichiometric Amount of NaSH Relative to OAHS

### Step 1.

O-Acetyl-L-homoserine was synthesized from L-homoserine and acetic anhydride according to the protocol described in the works of Sadamu Nagai, “Synthesis of O-acetyl-L-homoserine”, Academic Press (1971), vol. 17, p. 423-424.

### Step 2.

5.25 g/l of O-acetyl-L-homoserine originating from step 1), this product being dissolved in 140 ml of water, are introduced into a thermostatically controlled 250 ml glass reactor. The solution is brought to 37° C. with mechanical stirring. Next, a superstoichiometric amount of NaSH dihydrate is added to the reactor (X5, i.e. 15 g/l). The pH of the reaction medium is adjusted to a value of 6.5 and then 5 g/l of OAHS Sulfhydrylase and 0.4 g/l of pyridoxal phosphate cofactor are added to the reaction mixture. The pH is maintained at a setpoint value of 6.5 using an aqueous ammonia solution (4 M).

The analyses by potentiometry, HPLC and NMR reveal a gradual disappearance of the OAHS and the gradual appearance of several products over time. The predominant compound formed is L-homocysteine with a significant proportion of L-homocystine (L-4,4′-dithiobis(2-aminobutanoic acid)).

In these tests, homocysteine sulfide (L-homolanthionine) is not formed since it is not detectable in the final reaction medium even in trace form.

An analysis of the reaction mixture at the end time made it possible to show that all of the OAHS is consumed at the end of the reaction since it is not detectable even in the form of traces.

The molar selectivities (calculated according to example 1) obtained with respect to the transformed OAHS are as follows:
- 80% of L-homocysteine,
- 20% of L-homocystine (L-4,4′-dithiobis(2-aminobutanoic acid)).

The molar yield of the reaction in terms of L-homocysteine is then 80%.

### Example 3: Process According to the Invention for Synthesizing L-Homocysteine in the Absence of Oxygen and in the Presence of a Superstoichiometric Amount of NaSH Relativeto OAHS

### Step 1.

O-Acetyl-L-homoserine was synthesized from L-homoserine and acetic anhydride according to the protocol described in the works of Sadamu Nagai, “Synthesis of O-acetyl-L-homoserine”, Academic Press (1971), vol. 17, p. 423-424.

### Step 2.

Solutions of OAHS, of NaSH and of OAHS sulfhydrylase, and water are separately degassed beforehand by dinitrogen sparging at the reaction temperature (before mixing) so as to eliminate the presence of dissolved oxygen.

The reactor is also inertized under dinitrogen.

### Step 3.

5.25 g/l of O-acetyl-L-homoserine originating from step 1), this product being dissolved in 140 ml of water, are introduced into a thermostatically controlled 250 ml glass reactor. The solution is brought to 37° C. with mechanical stirring. Next, a superstoichiometric amount of NaSH dihydrate is added to the reactor (X5, i.e. 15 g/l). The pH of the reaction medium is adjusted to a value of 6.5 and then 5 g/l of OAHS Sulfhydrylase and 0.4 g/l of pyridoxal phosphate cofactor are added to the reaction mixture. The pH is maintained at a setpoint value of 6.5 using an aqueous ammonia solution (4 M).

The analyses by potentiometry, HPLC and NMR reveal a gradual disappearance of the OAHS and the gradual appearance of L-homocysteine. In these tests, homocysteine sulfide (L-homolanthionine) and the disulfide (L-homocystine) are not formed and are not detectable in the final reaction mixture.

An analysis of the reaction mixture at the end time made it possible to show that all of the OAHS is consumed at the end of the reaction since it is not detectable even in the form of traces.

A yield of L-homocysteine of around 100% is obtained.

1. Process for synthesizing at least one functionalized mercaptan of the following general formula (I): 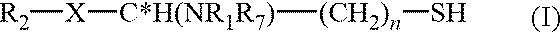
in which,
R₁ and R₇, which are identical or different, are a hydrogen atom or an aromatic or nonaromatic, linear, branched or cyclic, saturated or unsaturated, hydrocarbon chain of 1 to 20 carbon atoms which may comprise one or more heteroatoms;
X is chosen from -C(=O)-, -CH₂- or -CN;
R₂ is:
(i) either absent when X represents -CN,
(ii) or a hydrogen atom,
(iii) or -OR₃, R₃ being a hydrogen atom or an aromatic or nonaromatic, linear, branched or cyclic, saturated or unsaturated, hydrocarbon chain of 1 to 20 carbon atoms which may comprise one or more heteroatoms,
(iv) or -NR₄R₅, R₄ and R₅, which are identical or different, being a hydrogen atom or an aromatic or nonaromatic, linear, branched or cyclic, saturated or unsaturated, hydrocarbon chain of 1 to 20 carbon atoms which may comprise one or more heteroatoms;
n is equal to 1 or 2; and * represents an asymmetric carbon;
said process comprising the steps of:
a) provision of at least one compound of the following general formula (II): 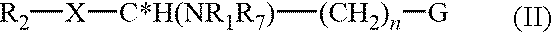
in which *, R
₁, R₂, R₇, X and n are as defined for formula (I) and
G represents either (i) R₆-C(O)-O-, or (ii) (R₇0)(R₈O)-P(O)-O-, or (iii) R₉O-SO₂-O-; with
R₆ being a hydrogen atom or a linear, branched or cyclic, saturated or unsaturated hydrocarbon chain of 1 to 20 carbon atoms which may comprise one or more aromatic groups and may be substituted by one or more groups chosen from -OR₁₀, (=O), -C(O)OR₁₁, -NR₁₂R₁₃;
R₁₀, R₁₁, R₁₂ and R₁₃ being independently chosen from:
H or a linear, branched or cyclic, saturated or unsaturated hydrocarbon chain of 1 to 20 carbon atoms;
R₇ and R₈, which are identical or different, being a proton, an alkali metal, an alkaline earth metal or an ammonium;
R₉ being chosen from a proton, an alkali metal, an alkaline earth metal or an ammonium;
b) provision of at least one hydrosulfide salt and/or sulfide salt or H₂S;
c) reaction between said at least one compound of formula (II) and said at least one hydrosulfide and/or sulfide salt or H₂S in the presence of at least one enzyme chosen from sulfhydrylases, and preferably a sulfhydrylase associated with said compound of formula (II); said reaction being performed essentially in the absence of oxygen, preferably in the absence of oxygen;
d) obtaining of at least one functionalized mercaptan of formula (I);
e) optional separation of said at least one functionalized mercaptan of formula (I) which is obtained in step d); and
f) optional additional functionalization and/or optional deprotection of the functionalized mercaptan of formula (I) which is obtained in step d) or e); and wherein steps a) and b) are optionally performed simultaneously.

2. Synthesis process according to claim 1, wherein step c) takes place in a reactor in which the reaction mixture comprises less than 0.0015% oxygen by weight relative to the total weight of the reaction mixture and/or the gas phase contained in the gas headspace of the reactor comprises less than 21% oxygen by volume relative to the total volume of said gas phase.

3. Synthesis process according to claim 1, wherein the hydrosulfide salt and/or sulfide salt or H₂S is in excess relative to the compound of formula (II), preferably during step c).

4. Synthesis process according to claim 1, wherein the molar ratio [hydrosulfide salt and/or sulfide salt] / [compound of formula (II)] or H₂S/compound of formula (II) is comprised between 1.5 and 10, preferentially between 2 and 8, and even more preferentially between 3.5 and 5, limits included, preferably during step c).

5. Synthesis process according to claim 1, wherein said at least one hydrosulfide and/or sulfide salt is chosen from the group consisting of: ammonium hydrosulfide, alkali metal hydrosulfides, alkaline earth metal hydrosulfides, alkali metal sulfides and alkaline earth metal sulfides.

6. Synthesis process according to claim 1, wherein the pH of the reaction medium in step c) is comprised between 4 and 9, for example between 5 and 8, preferably between 6 and 7.5, and more particularly between 6.2 and 7.2, limits included.

7. Synthesis process according to claim 1, wherein the compound of formula (II) is chosen from the group consisting of: O-phospho-L-homoserine, O-succinyl-L-homoserine, O-acetyl-L-homoserine, O-acetoacetyl-L-homoserine, O-propio-L-homoserine, O-coumaroyl-L-homoserine, O-malonyl-L-homoserine, O-hydroxymethylglutaryl-L-homoserine, O-pimelyl-L-homoserine and O-sulfato-L-homoserine, preferably O-acetyl-L-homoserine.

8. Process according to claim 1, wherein said compound of formula (II) is O-acetyl-L-homoserine, the enzyme used is O-acetyl-L-homoserine sulfhydrylase and the functionalized mercaptan of formula (I) is L-homocysteine.

9. Process according to claim 1, wherein said sulfhydrylase is a transferase of the E.C.2. type.

10. Composition, preferably solution, comprising:
a compound of formula (II) as defined in claim 1;
a sulfhydrylase, preferably a sulfhydrylase associated with the compound of formula (II); and
ammonium hydrosulfide NH₄SH or H₂S in excess.
